(19) United States
(12) Patent Application Publication
(10) Pub. No.: US 2012/0195953 A1
(43) Pub. Date: Aug. 2, 2012

(54) Fibrin sealant (FIBRINGLURAAS) consisting of a kit of lyophilized high concentrate fribinogen intentionally enriched and preserved with fibronolysis inhibitor A1AT

(76) Inventor: Kieu Hoang, Agoura Hills, CA (US)

(21) Appl. No.: 13/108,970

(22) Filed: May 16, 2011

Related U.S. Application Data
(63) Continuation-in-part of application No. 13/064,070, filed on Mar. 4, 2011, now abandoned.
Application No. 13/064,070 is a continuation-in-part of application No. 11/990,203, filed on Jul. 15, 2008, now abandoned.
Application No. 11/990,203 is a continuation-in-part of application No. 12/457,796, filed on Jun. 22, 2009, now Pat. No. 8,013,122.
Application No. 12/457,796 is a continuation-in-part of application No. PCT/US2007/020258, filed on Sep. 19, 2007.
(60) Provisional application No. 61/457,380, filed on Mar. 14, 2011.
(60) Provisional application No. 61/452,860, filed on Mar. 15, 2011.
(60) Provisional application No. 61/472,930, filed on Apr. 7, 2011.

(51) Int. Cl. A61K 9/70; C12N 9/74; A61P 27/00; A61P 35/00; A61P 17/00; A61K 38/48; C07K 14/75

(52) U.S. Cl. 424/443; 424/94.64; 435/214; 530/382

(57) ABSTRACT

Fibrin sealant (FIBRINGLURAAS®) consisting of a kit of lyophilized high concentrate fribinogen intentionally enriched and preserved with fibronolysis inhibitor A1AT, either non-heated or heating to at least 1° C. and above, preferably at least 101° C., and lyophilized thrombin used to compound glue membrane, the diameter of which is less than 10 micrometers the actual size of the glue membrane of the fibrin sealant (FIBRINGLURAAS®) is from 0.6 μm, to 101° C. heating 0.005 micrometers. Thrombin, a protein, contains good healthy cells. High concentrate fibrinogen, another protein, contains good healthy cells. AFOD (HDL ApoA1), another protein, contains good healthy cells and its topical applications for all solid tumor cancers.

### CROSS REFERENCE TO RELATED APPLICATIONS

This application is a continuation-in-part of prior application No. 13/064,070, filed Mar. 4, 2011, which is hereby incorporated herein by reference in its entirety and which is a continuation-in-part of prior application Ser. No. 11/990,203, filed Jul. 15, 2008, which is hereby incorporated herein by reference in its entirety. The benefit under 35 USC 120 is hereby claimed of the filing dates of provisional application No. 61/457,380, filed on Mar. 14, 2011 and provisional application No. 61/457,380, filed on Mar. 14, 2011 and provisional application No. 61/452,860, filed on Mar. 15, 2011 and provisional application No. 61/472,930, filed on Apr. 7, 2011, all of which are hereby incorporated herein by reference in their entireties.

Fibrin sealant (FIBRINGLURAAS®) consisting of a kit of lyophilized high concentrate fribinogen intentionally enriched and preserved with fibronolysis inhibitor A1AT, either non-heated or heating to at least 1° C. and above, preferably at least 101° C., and lyophilized thrombin used to compound glue membrane, the diameter of which is less than 10 micrometers the actual size of the glue membrane of the fibrin sealant (FIBRINGLURAAS®) is from 0.6 μm, to 101° C. heating 0.005 micrometers.

Thrombin, a protein, contains good healthy cells. High concentrate fibrinogen, another protein, contains good healthy cells. AFOD (HDL ApoA1), another protein, contains good healthy cells and its topical applications for all solid tumor cancer.

### DESCRIPTION OF INVENTION AND ITS PURPOSES

1. A kit of lyophilized high concentrated fibrinogen (hcfng) intentionally enriched and preserved with the fibrinolysis inhibitor alpha 1-antitrypsin (A1AT) and either not heated or dry, wet or vapor heated up to at least 1° C., preferably at least 101° C., during the purification process of the high concentrated fibrinogen (HcFNG) which is used in the kit of Fribin Sealent (FinbinGluRAAS is much different than a regular fibrinogen under the trade name “FibroRAAS®.

High concentrate Fibrinogen contains Factor XIII, clottable fibrinogen purity should be

EQUAL to or HIGHER than 80%. Clotting activity should be less or equal to 60 seconds.
And concentration range from 5% to 9% (FIVE to NINE PERCENT) and should be TOPICAL USE whereas regular Fibrinogen (FibroRAAS®) CAN BE INJECTED and need an osmotic pressure equal or greater than 240 mOsmol/kg and purity is only equal or less than 70% and Concentration is ONLY 2% (TWO PERCENT)
FIBRIN SEALANT (FibrinGluRAAS®) has been used as TOPICAL HEMOSTASIS drug in the treatment of the surface of burns, abdominal incisions of general surgery, oozing of blood in liver operations, and blood vessel surgery to stop bleeding.
FIBRINOGEN FibroRAAS® can be INJECTED and use in the treatment of:
1. Congenital hypofibrinogenaemia or fibrinogenaemia.
2. Acquired hypofibrinogenaemia; severe liver damage, cirrhosis, disseminated intravascular coagulation, disorder of blood coagulation due to the lacking fibrinogen caused by obstetric hemorrhage, big surgical, trauma or internal hemorrhage.
   A kit of lyophilized High concentrate Fibrinogen described above and lyophilized thrombin is used to compound glue membrane, the kit and the glue membrane both having the name FibrinGluRAAS®, the diameter of the glue membrane mesh is smaller than human cancer cells, which are of the size of 10-100 μm. Preferably, the glue membrane mesh is 0.6 μm or less in its biggest dimension, and far smaller than human tumor cells of 10-100 μm, which are at least 15 fold bigger than the fibrin mesh formed. The glue membrane can prevent cancer cells from becoming detached and spreading into the abdominal cavity during the surgical operations of gastrointestinal cancer in mice. Such a glue membrane has clinical applications other than gastric and gastrointestinal cancer in humans, such as colon and breast cancer, and all kinds of solid tumors that have not yet spread to other parts of body, such as AIDS related cancers, osteosarcoma, and cancers of the anus, appendix, bile duct, bladder, brain, breast, cervix, colon, esophagus, eye, gall bladder, head, neck, heart, liver, kidney, larynx, lip, oral cavity, lung, mouth, paranasal sinus and nasal cavity, ovaries, pancreas, parathyroid, penis, prostate, rectum, salivary glands, skin, spleen, throat, testicles, urethra, and vagina, as well as renal cell carcinoma.

2. The compound for the glue membrane according to the present invention should be applied TOPICALLY and SHOULD NOT BE INJECTED and SHOULD NOT APPLY for leukemias, including acute myeloid leukemia (M0-M7), lymphoma, marrow malignancy, acute lymphoid leukemia (small, middle, large), myeloid disfunction syndrome (MDS), anemia, lupus and sclerosis in the brain. (Another RAAS ATBC 1-9 Agents under a different Application)

3. If USED ALONE, the fibrin sealant membrane can trap the cancer cells. This means that the fibrin sealant membrane separates tumor cells from healthy tissue, and that blood vessels cannot reach cancer cells, so that theoretically the fibrin sealant membrane (FibrinGluRAAS®) can INHIBIT cancer cells from releasing cytokines, including TNF (Tumor Necrosis Factor), and activation of histones to healthy tissues. In other words, the fibrin sealant membrane, if USED ALONE, can inhibit the toxicity produced by only ONE TIME radio-chemo therapy because the fibrin sealant membrane can trap cancer cells and hold them back, so that it prevents the invasion of tumor cells into healthy tissue. The fibrin sealant membrane (FibrinGluRAAS®) can be used as an adjuvant instrument to prevent cancer cells from becoming detached and spreading into the abdominal cavity during surgical operations of gastrointestinal cancer.

4. The high concentrated fibrinogen, which has the name FibrinGluRAAS®, can be combined with agent such as fluorouracil (C4H3FN2O2), available under the name RAAS 1 to 45 FU, which can KILL the tumor cells. With this combination, the overall effect is that the GROWTH of TUMORS will be INHIBITED. Fluorouracil is a pyrimidine analog which is used in the treatment of cancer. It works through noncompetitive inhibition of thymidylate synthase.

It was designed, synthesized and patented by Charles Heidelberger in 1957. When flourouracil (C4H3FN202) is applied together with the fibrin sealant membrane in vivo, the MOLECULES of flourouracil are also trapped in the fibrin sealant membrane (FibrinGluRAAS®), which keeps the drug molecules stored in the membrane so that they can be released slowly. This effect also makes the DRUG much more CONCENTRATED LOCALLY and less TOXIC, when compared with injecting the drug INTRAVENOUSLY. Fluorouracil is just one agent that can KILL the tumor cells and perform other tasks as described above. Other agents that can KILL the tumor cells can be used instead of, or in addition to, fluorouracil. The agents that can KILL the tumors that are contemplated for use with the present invention include all presently known such agents, as well as all such agents that will become available in the future. In order to prevent TOXICITY produced by ONLY A ONE TIME CHEMO-RADIO THERAPY immediately Follow by a surgical operations to REMOVE Cancer Tumors, then FibrinGluRAAS® can be applied TOPICALLY ALONE or in combination with fluorouracil (C4H3FN202), which can KILL ANY REMAINING Cancer Tumor cells after the surgical operations.

5. If FibrinGluRAAS® is used ALONE, THROAT Cancer patients will NOT LOSE THEIR TASTE, because No Chemo-Radio Therapy is USED.

6. Cancer cells may secrete TNF, various other cytokines, special proteins, even proteinase or some special hormones, depending on the type of cancer. For example, some liver cancer cells secrete alpha fetal protein. Some breast cancer, lung cancer and prostate cancer cells can release cytokines like interleukins. As the cancer cells are trapped in the fibrin sealant membrane and later killed by chemotherapy reagents combined with the fibrin sealant, secreted TNF, various other cytokines, special proteins, even proteinase or some special hormones can also be trapped in the fibrin sealant membrane. A1AT in the fibrin sealant INHIBITS the ACIVITY of SOME PROTEINASE secreted by Cancer cells or nearby healthy cells.

### BACKGROUND OF THE INVENTION

A. Field of Invention

The present invention relates to a kit of lyophilized thrombin and lyophilized high concentrated fibrinogen and its usage to prevent tumor cell pervasion caused by incision and trauma in tumor operations. The present invention addresses a number of problems. For example, in tumor operations, incision and trauma usually cause tumor cell pervasion that can increase the risk of tumor palindromia and metabasis after tumor operations and shorten the life span of the patients.

A glue membrane made from a compound of the high concentrated fibrinogen and thrombin, according to the present invention, is applied to prevent pervasion of tumor cells caused by incision and trauma in a tumor surgical operation. The glue membrane can reduce the risk of palindromia and metabasis of the tumor after the operation and INCREASE the LIFE SPAN of the patient. It is possible that, due to the glue membrane to stop the dissemination of tumor cells, cancers will not reoccur.

The high concentrated fibrinogen, which has the name FibrinGluRAAS®, can be combined with a agent such as fluorouracil (C4H3FN2O2), available under the name RAAS 1 to 45 FU, as an adjuvant instrument to prevent cancer cells from becoming detached and spreading into the abdominal cavity during surgical operation of gastrointestinal cancer, and the fluorouracil in the resultant glue membrane will KILL THE TUMOR CANCER CELLS.

There is a tendency for lumps to develop where the mamma or a lump has been removed, after radical mamma and lump exsection. Daubing the high concentrated fibrinogen and thrombin that form the glue membrane according to the present invention in the places of removal of the mamma or a lump reduces the number and/or size of lumps that tend to form there.

A compound of lyophilized thrombin and high concentrated fibrinogen has also been used as a topical hemostasis drug in the treatment of the surface of burns, abdominal incisions of general surgery, oozing of blood in liver operations, and blood vessel surgery.

### SUMMARY OF THE INVENTION

In accordance with the present invention, a compound of lyophilized thrombin and high concentrated fibrinogen intentionally enriched and preserved with the fibrinolysis inhibitor alpha 1-antitrypsin (A1AT) and either not heated or dry, wet or vapor heated up to at least 1° C., preferably at least 101° C., during the purification process of the high concentrated fibrinogen is used to compound a glue membrane that is used to prevent dissociative tumor cell pervasion. The A1AT is enriched and preserved during the purification process by dialysis to concentrate the A1AT up to 5% to 9% whereas Regular fibrinogen is concentrated up to 2%, and pure A1AT is added to the product to maximize the stability and density of the membrane.

A kit of lyophilized thrombin and high concentrated fibrinogen in accordance with the present invention produces a solid-meshy glue membrane to prevent dissociative tumor cell pervasion and to prevent cancer cells from becoming detached and spreading into the abdominal cavity during surgical operations of gastrointestinal cancer in mice and other clinical applications, such as colon and breast cancer, and it can be used for all kinds of solid tumors.

Due to the purification process of the high concentrated fibrinogen according to the present invention, the diameter of the glue membrane mesh is smaller than human cancer cells, which are of the size of 10-100 μm. Preferably, the glue membrane mesh is 0.6 μm which is 15 folds less and far smaller than tumor cells of 10-100 μm.

FibrinGluRAAS® can inhibit the release of cytokines, including TNF (Tumor Necrosis Factor), stop the activation of histones and toxicity produced by radio-chemo therapies to prevent the deaths of cancer patients due to the release of cytokines, TNF and stop the activation of histones and toxicity produced by radio-chemo therapies if USED ALONE. The high concentrated fibrinogen, which has the name FibrinGluRAAS®, can be combined with an agent such as fluorouracil (C4H3FN2O2), available under the name RAAS 1 to 45 FU, as an adjuvant instrument to prevent cancer cells from becoming detached and spreading into the abdominal cavity during surgical operation of gastrointestinal cancer, and the fluorouracil in the resultant glue membrane will KILL THE TUMOR CANCER CELLS.

### BRIEF DESCRIPTION OF THE DRAWINGS

Other objects and features of the present invention will become apparent from the following detailed description considered in connection with the accompanying drawings. It should be understood, however that the drawings are designed for the purpose of illustration only and not as a definition of the limits of invention.

FIG. 1 and FIG. 2 are electron micrographs of a glue membrane compounded by daubing a solution of lyophilized thrombin and a solution of lyophilized high concentrated fibrinogen one layer after another on a glass slide in connection with an IN VITRO study.

FIG. 3A1 and FIG. 3A2 are electron micrographs of a control without a glue membrane.

FIGS. 3B1 and 3B2 are electron micrographs of a material surface with a glue membrane according to the present invention in a tumor cell pervasion experiment.

FIG. 3C1 and FIG. 3C2 are electron micrographs of a glue membrane in a tumor cell pervasion experiment involving a glue membrane treatment according to the present invention.

### DETAILED DESCRIPTION OF THE PREFERRED EMBODIMENTS

The effects of fibrinogen and thrombin on the process of thrombosis are well known.

There are many products from fibrinogen and thrombin, but the products are used to stanch after operations in most cases.

According to the present invention, a solid-meshy membrane compounded by lyophilized thrombin and high concentrated fibrinogen (FinbrinGluRAAS®) that is intentionally enriched and preserved with the fibrinolysis inhibitor alpha 1-antitrypsin (A1AT) during the purification of the high concentrated fibrinogen and either not heated or dry, wet or vapor heated up to at least 1° C., preferably at least 101° C., to intensify the stability and durability of the high concentrated fibrinogen has a mesh that is smaller than human cancer cells, which are of the size of 10-100 μm. Preferably, the glue membrane mesh is 0.6 μm in its biggest dimension, and far smaller than human tumor cells of 10-100 μm.

The high concentrated fibrinogen and thrombin are sourced from human plasma tested by nucleic acid testing (NAT) to be negative for HIV 1&2, hepatitis B (HBV) and hepatitis C (HCV). Both enveloped viruses and non-enveloped viruses are inactivated by a solvent detergent step (S/D) using tri(n-butyl)phosphate (TNBP) and Tween 80; nanofiltration is used to remove both enveloped viruses and non-enveloped viruses for the thrombin; and either not heating or dry, wet or vapor heating up to at least 1° C., preferably at least 101° C. for 30 minutes is done to inactivate the non-enveloped viruses for the high concentrated fibrinogen. ALL viruses, both enveloped and non-enveloped, are inactivated by these steps. Both components, high concentrated fibrinogen and thrombin, are lyophilized and put into separate solutions.

### In Vitro Study:

In experiments using the present invention, a thin and smooth layer of high concentrated fibrinogen solution is daubed on the surface of a glass slide, or on the bottom surfaces of cell culture inserts in an assay kit, to form a coating. After about 5 seconds, a thin and smooth layer of thrombin solution is daubed sequentially on the high concentrated fibrinogen coating. The daubing of the high concentrated fibrinogen solution and then the thrombin solution is repeated about 3-5 times. A solid-meshy glue membrane forms quickly. The diameter of the glue membrane mesh is smaller than human cancer cells, which are of the size of 10-100 μm. Preferably, the glue membrane mesh is 0.6 μm or less in its biggest dimension, as little as 0.0 μm, and far smaller than human tumor cells of 10-100 μm. The glue membrane can hold back the human tumor cells and prevent pervasion.

Thus, the high concentrated fibrinogen and thrombin solutions are daubed alternately with one another on the local surface of tumor tissue, one layer at a time. The solid-meshy glue membrane that forms on the local tissue surface prevents dissociative tumor cell pervasion in operations, reduces the risk of palindromia and metabasis of tumors after treatment, and improves the life span of patients, and possibly cancer will not reoccur. The glue membrane has a good biological compatibility and convenient usage.

### Example 1

Generation of Glue Membrane

(1) The high concentrated fibrinogen solution and the thrombin solution of the kit according to the present invention were each daubed three times, alternately, one layer at a time, on a 0.8 cm×0.8 cm slide surface (compounded by 450 IU thrombin and 40 mmol/L CaCl₂).

(2) The layers of the high concentrated fibrinogen solution and thrombin solution were air dried and inspected with an electron microscope.

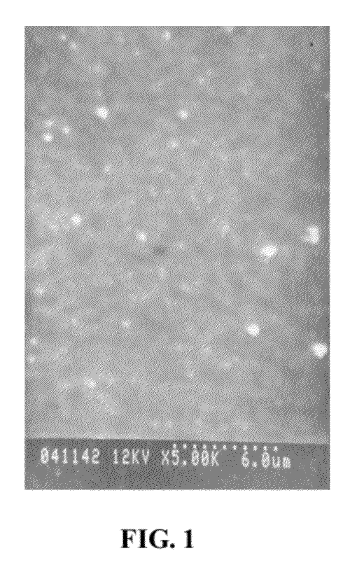

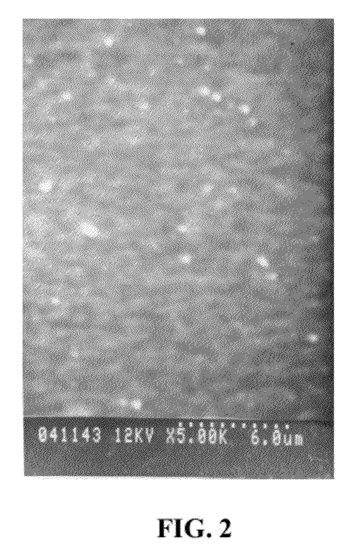

FIG. 1 and FIG. 2 are electron microscope photographs of the dried high concentrated high concentrated fibrinogen and thrombin layers. The amplification is 5000.

From these photographs, the smooth glue membrane surface can be seen. There are no distinct holes in the glue membrane. It can be induced from the amplified scale that the mesh bore diameter is 0.6 μm or less, as little as 0.0 μm. Human cancer tumor cell size is 10-100 μm. Thus, the glue membrane compounded by human plasma high concentrated fibrinogen and thrombin can HOLD BACK human TUMOR CELLS and PREVENT PERVASION.

### Example 2

From a glue membrane kit according to the present invention for preventing dissociative tumor cell pervasion:

(1) A thin and smooth glue membrane was produced on the bottom surfaces of the cell culture inserts that are placed into the wells of the tissue culture plate of a cell invasion assay kit ECM550, commercially available from Chemicon International of Temecula, Calif., to form a coating thereon by using the method of EXAMPLE 1. Then, a cell invasion experiment was carried out according to the instructions provided in the cell invasion assay kit. The invasive cells in the cell suspensions that were placed in the inserts included carcinoma ventriculi cell lines (tumor of the stomach), human gastric adenocarcinoma cell line KN45, and AGS human cultured gastric adenocarcinoma cells from Ruijing Hospital of Shanghai, China, as well as human breast cancer cells MDA-MB-231 and colon cancer cells Ls 174T from SBI (System Biosciences of Mountain View, Calif.).

(2) In accordance with the instructions in the cell invasion assay kit, the spent medium was discarded, the inner membrane was cleared using a cotton-tipped swab, and the inserts were stained for 20 minutes and air dried. There was no high concentrated fibrinogen or thrombin in the control wells of the tissue culture plate.

See FIGS. 1 and 2.

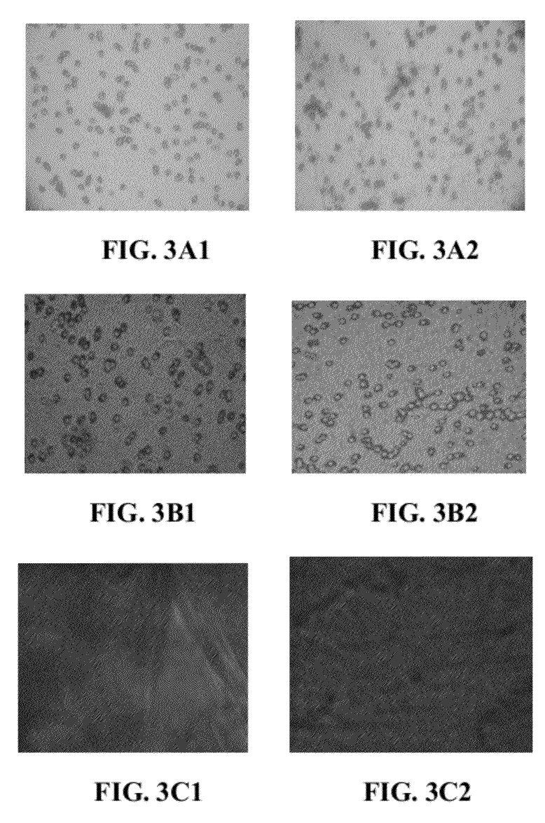

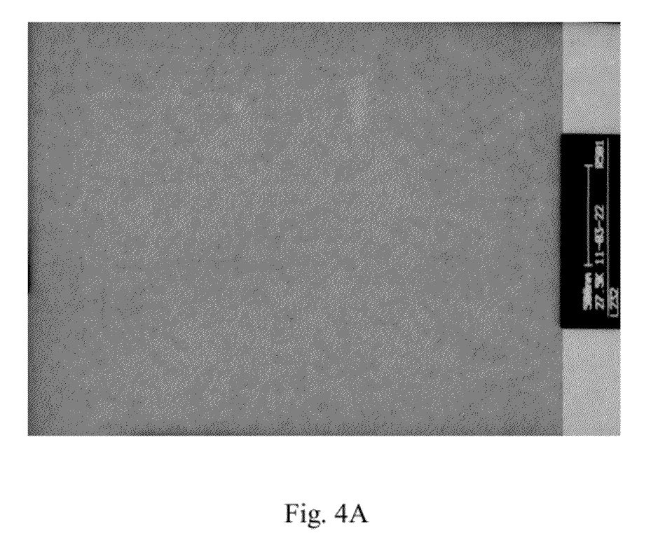

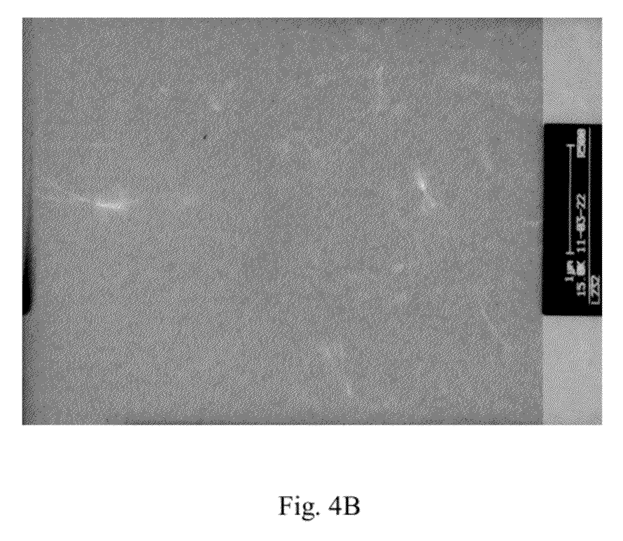

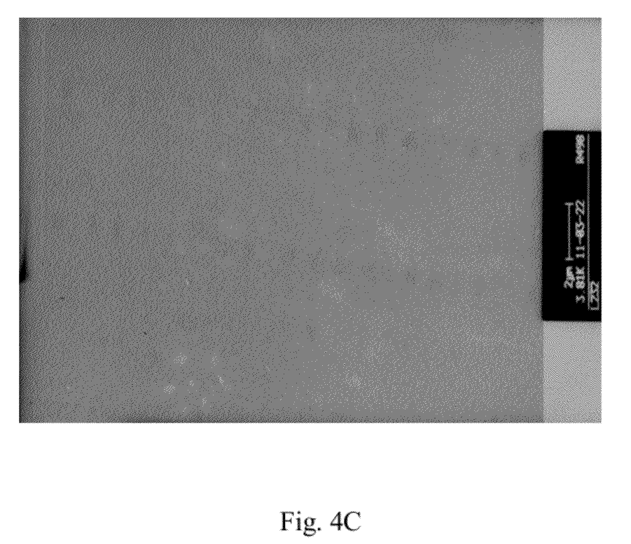

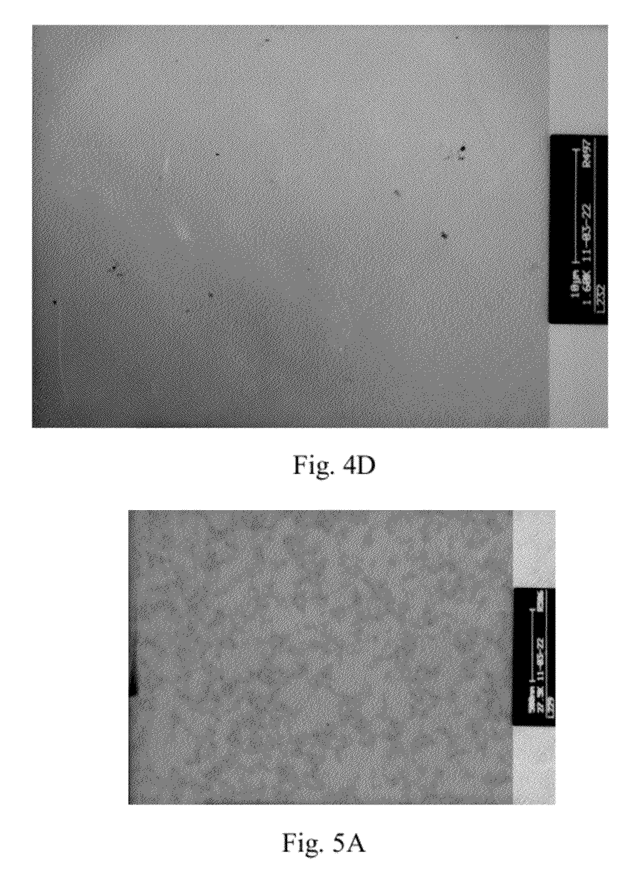

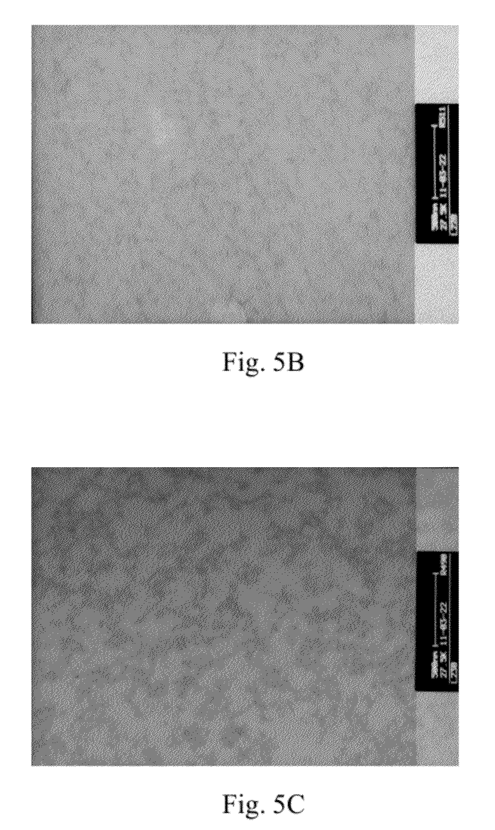

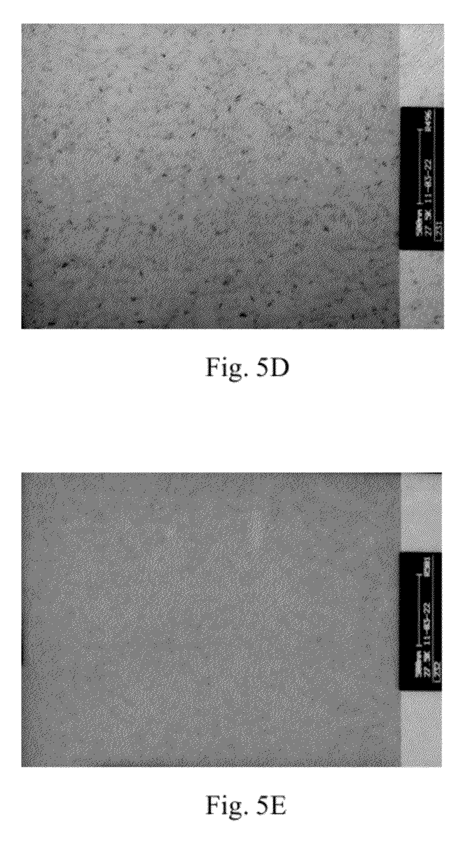

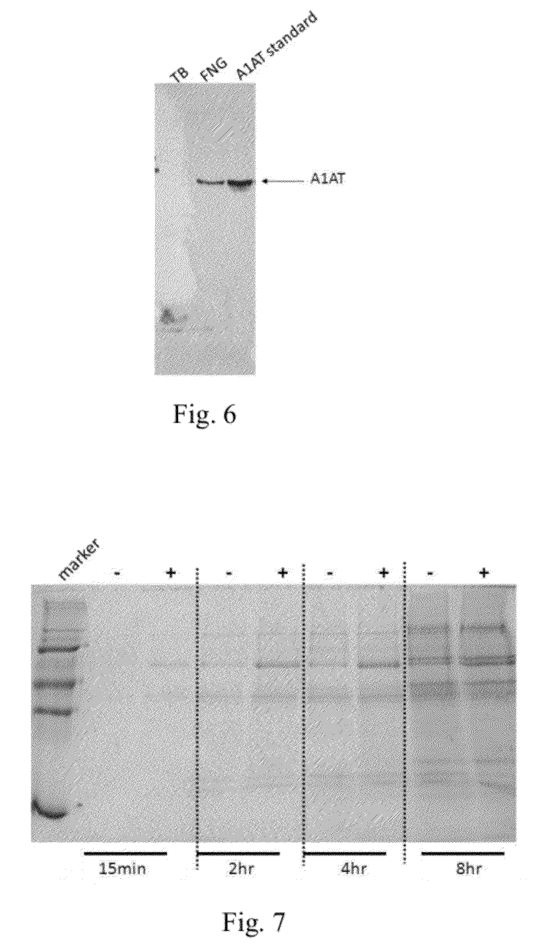

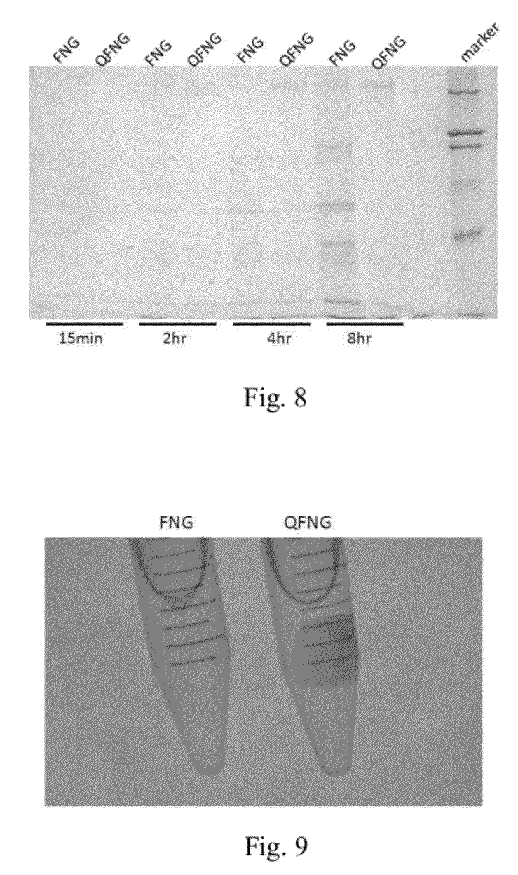

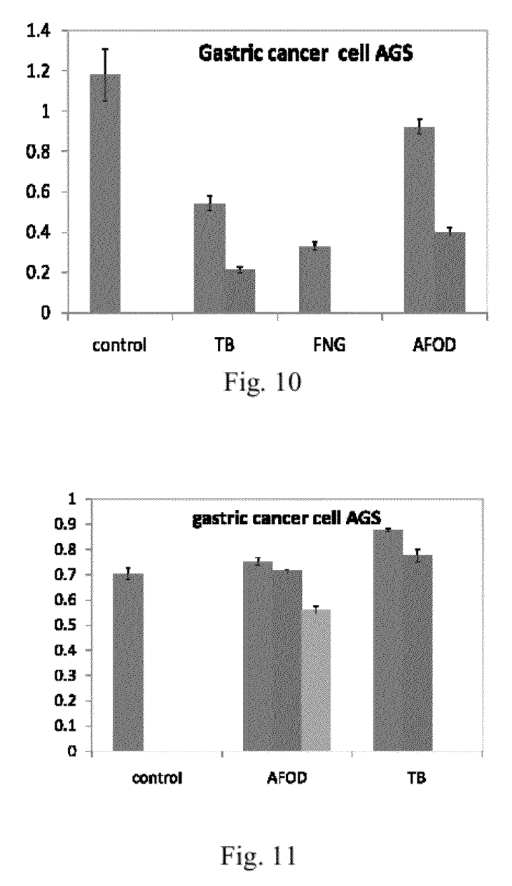

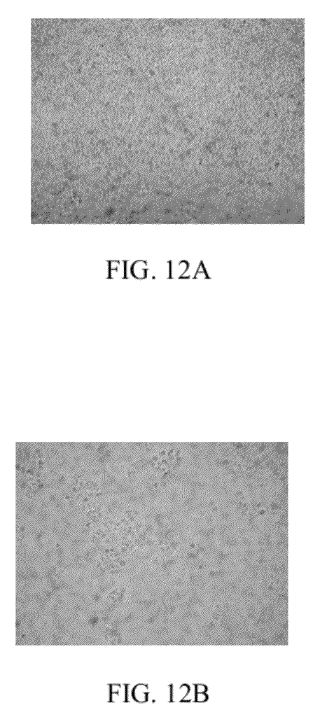

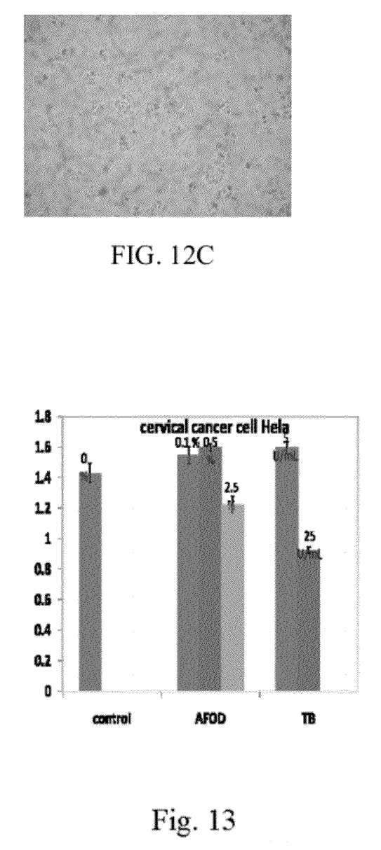

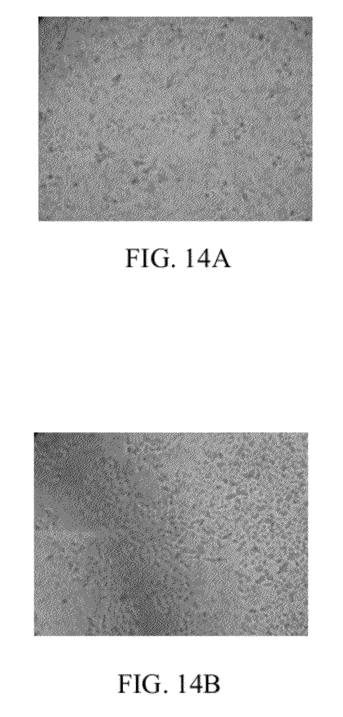

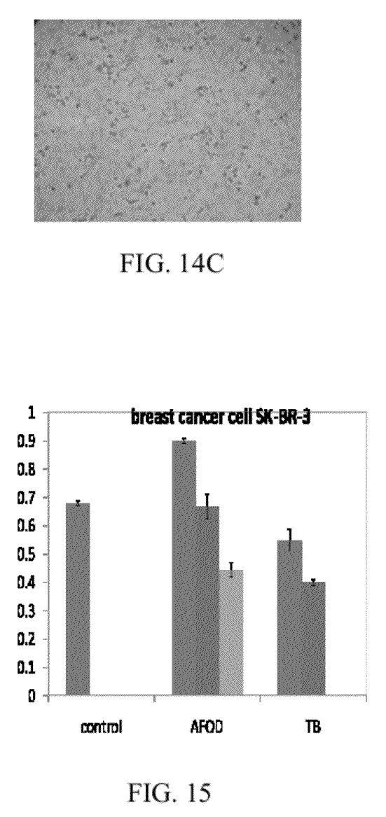

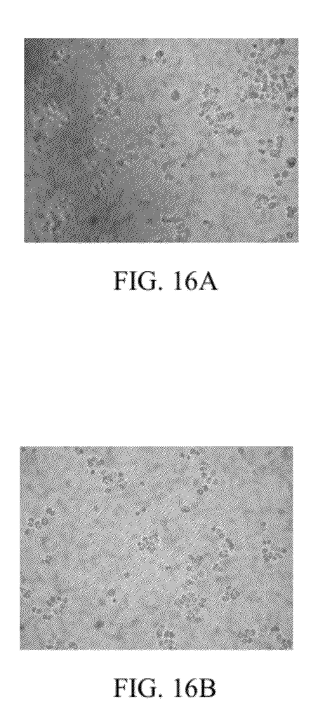

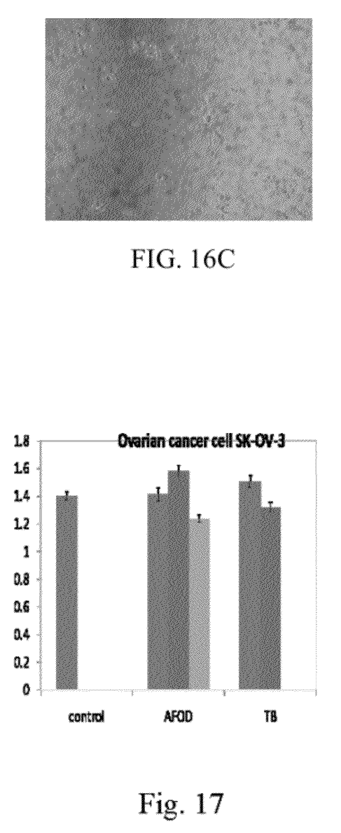

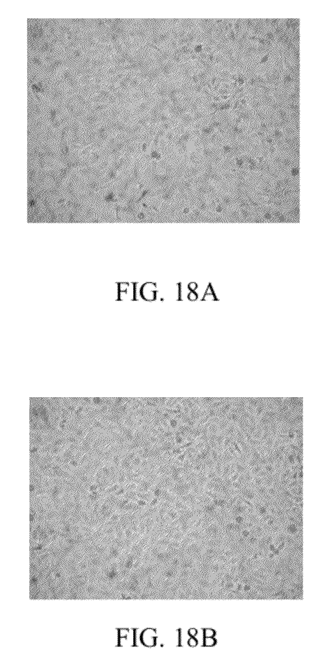

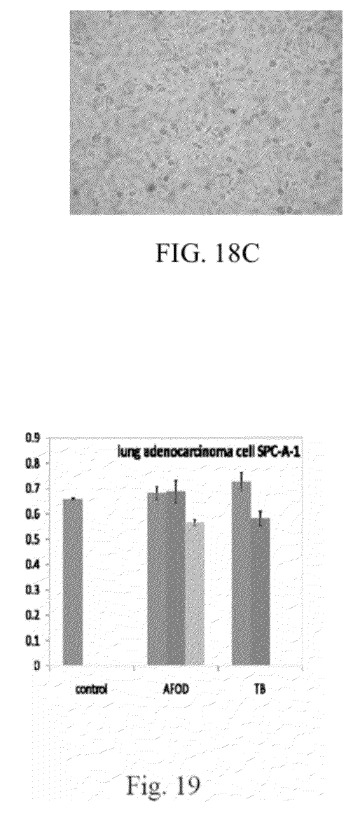

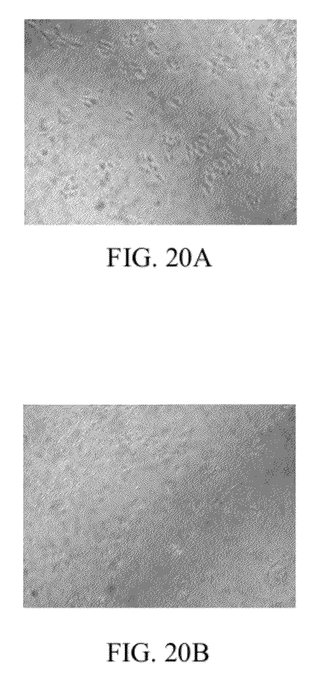

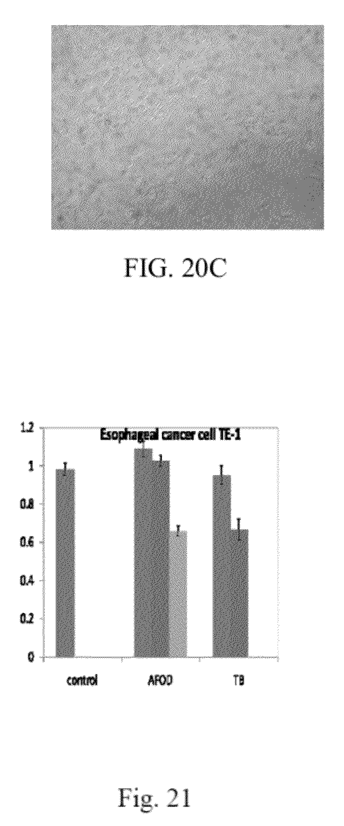

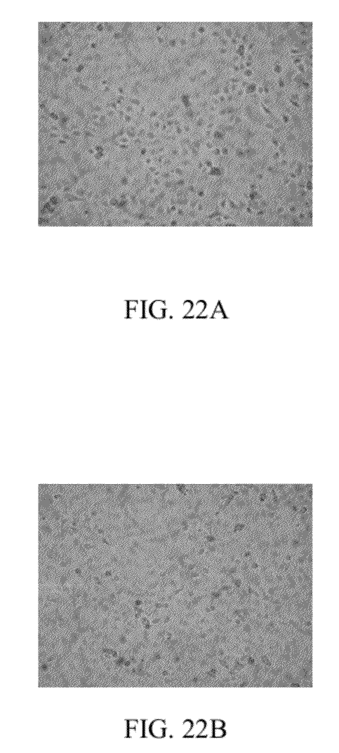

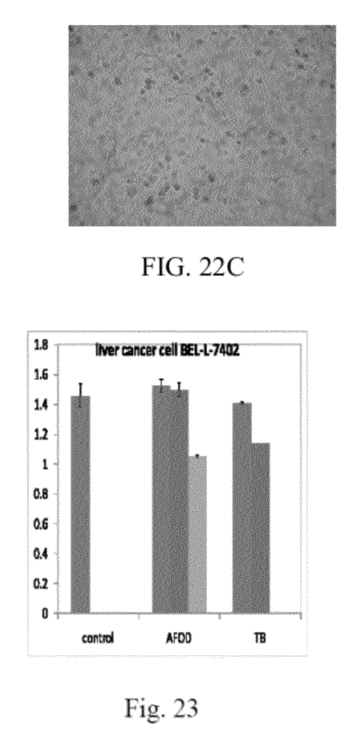

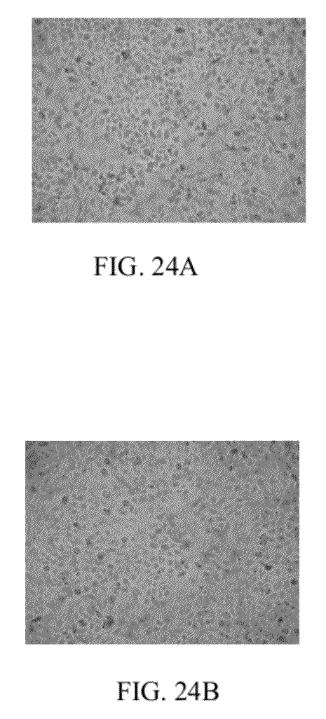

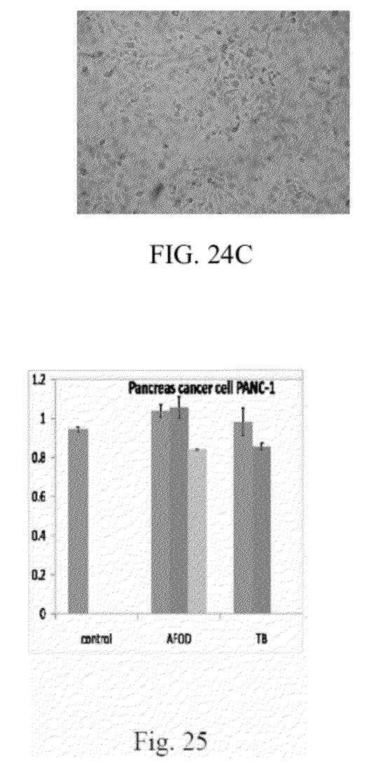

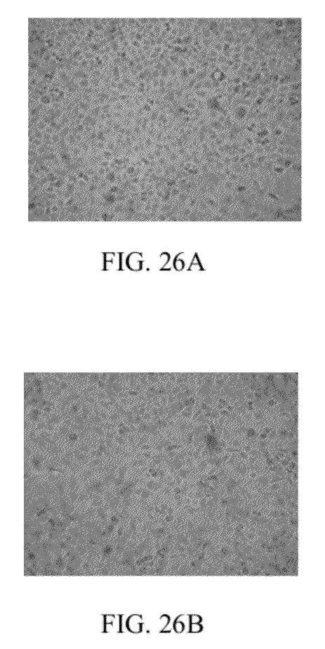

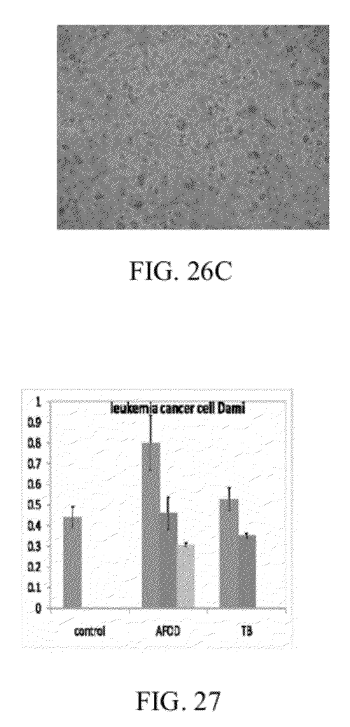

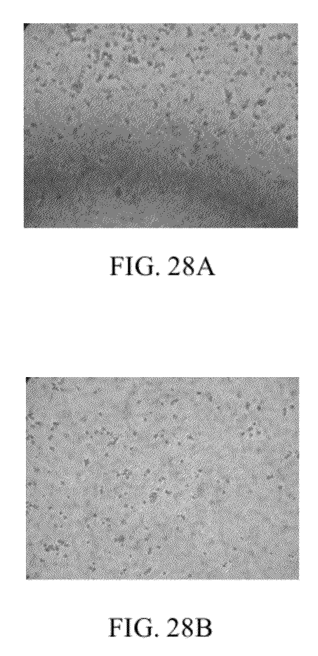

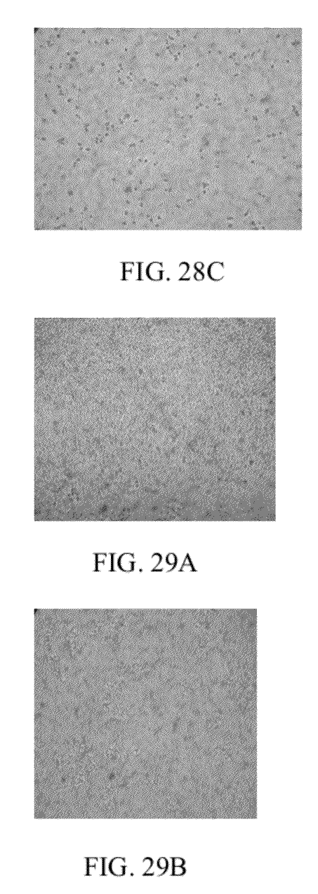

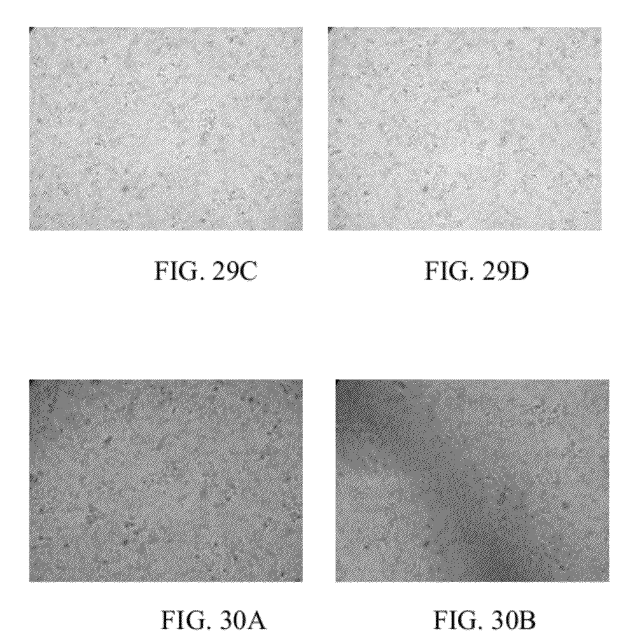

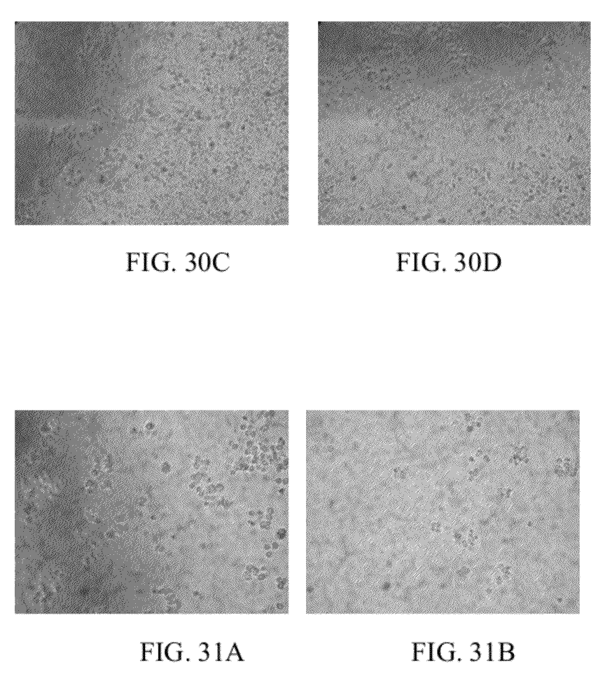

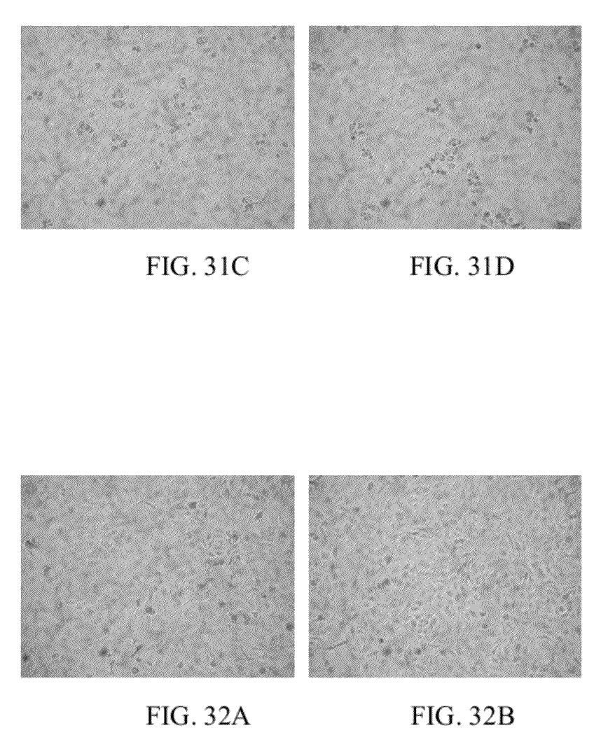

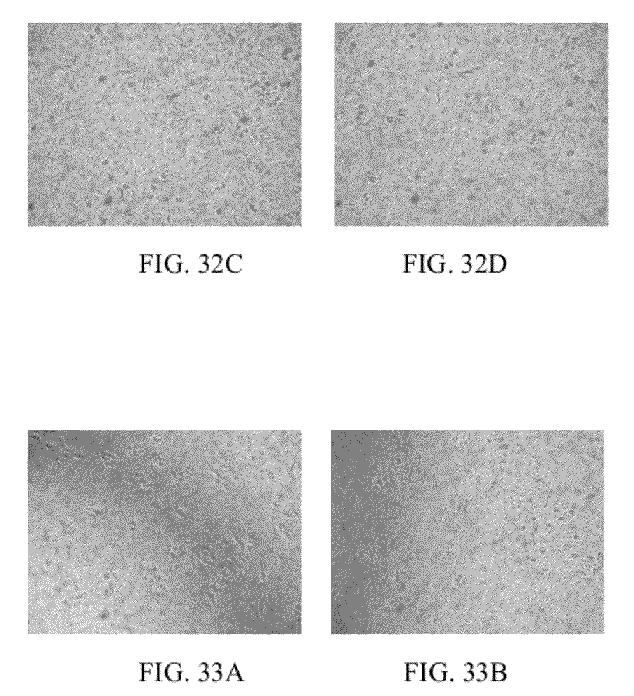

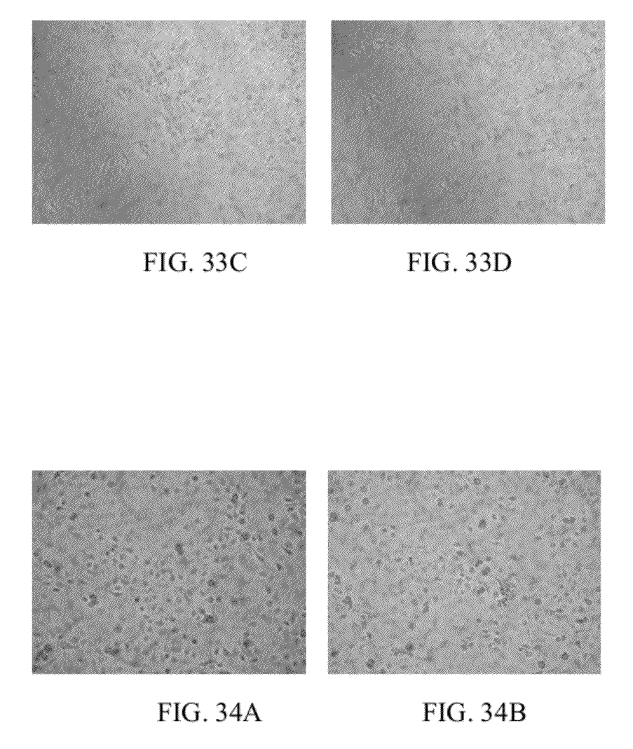

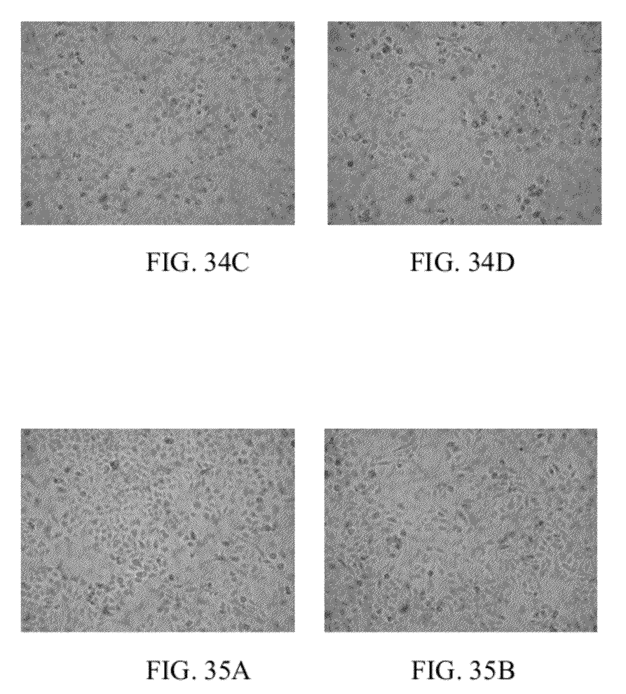

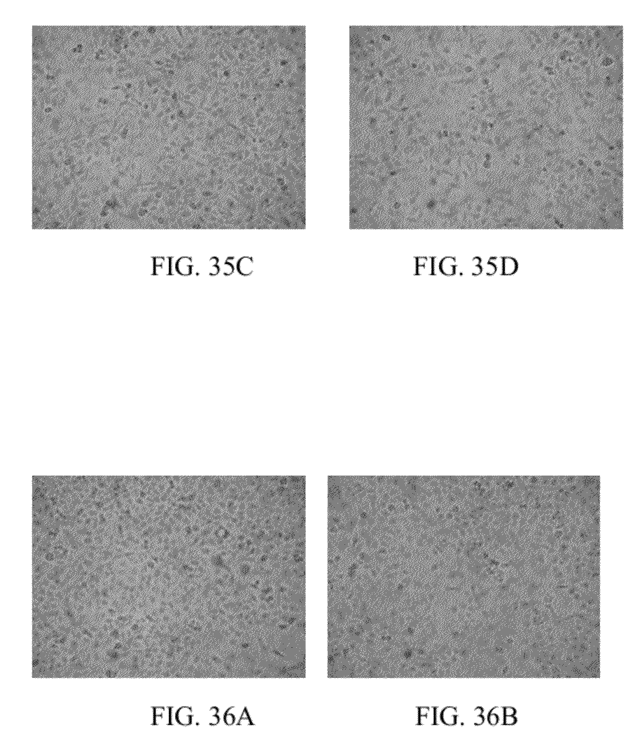

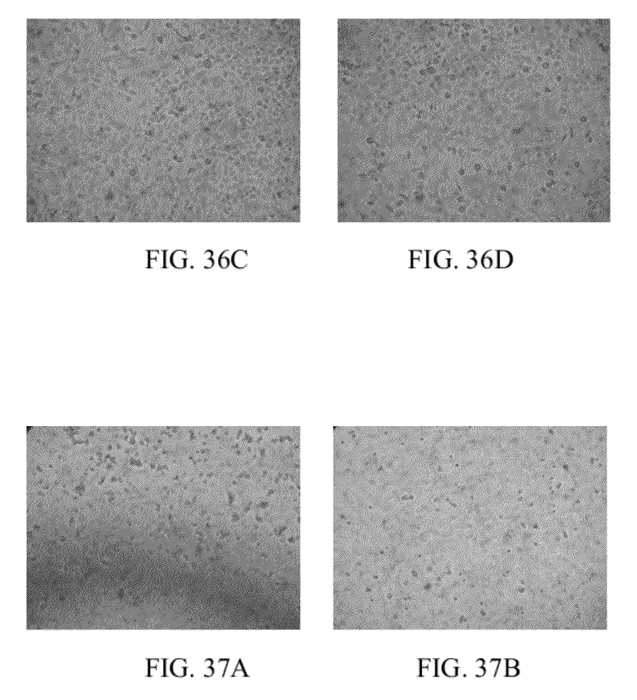

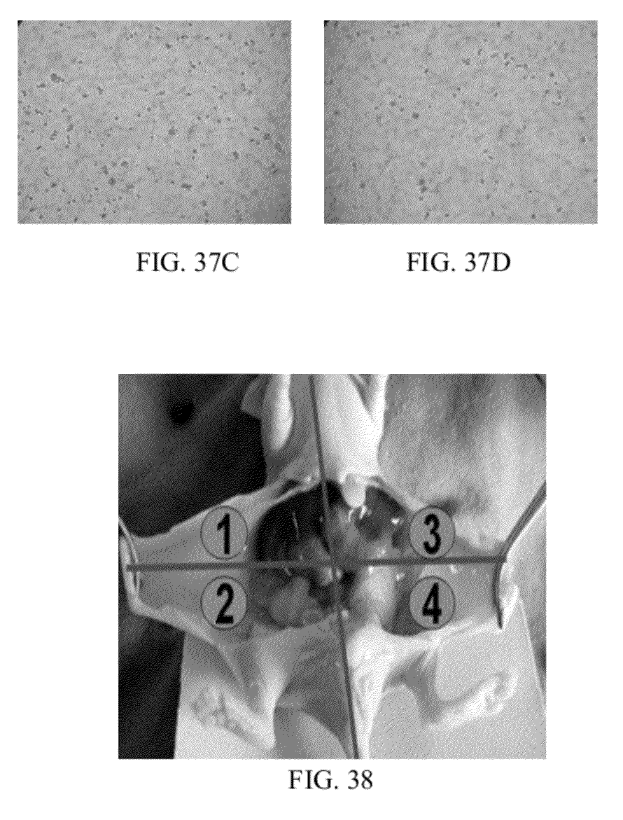

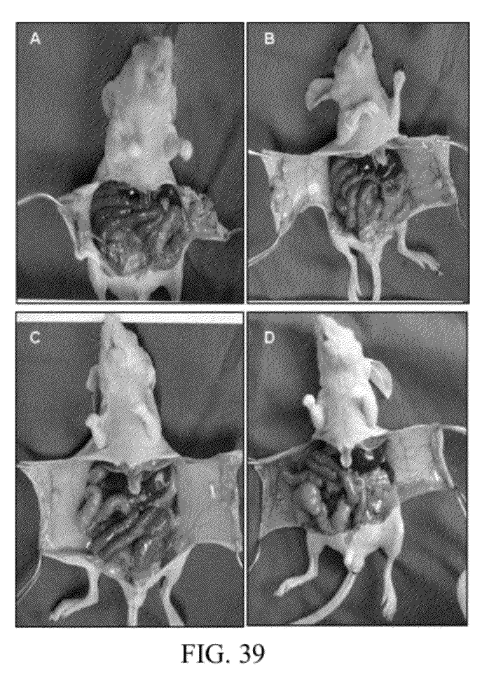

(3) Electron microscope photographs were taken, including the electron microscope photographs of FIG. 3A1, FIG. 3A2, FIG. 3B1, FIG. 3B2, FIG. 3C1 and FIG. 3C2, and records were made.

FIG. 3A1, FIG. 3A2, FIG. 3B1, FIG. 3B2, FIG. 3C1 and FIG. 3C2 show that the glue membrane of EXAMPLE 2 arrested dissociative tumor cells in the inserts and prevented tumor cell pervasion through the glue membrane and, therefore, also support the conclusion that the glue membrane can hold back human tumor cells and prevent pervasion.

FIGS. 3A1 and 3A2 are electron microscope photographs without a glue membrane showing that cell invasion occurs where there is no glue membrane. FIGS. 3B1 and 3B2 are photographs of prevention of cell invasion on the inner side of the glue membrane. FIGS. 3C1 and 3C2 are photographs of the prevention of cell invasion on the exterior side of the glue membrane. It is very clear that glue membrane compounded by human plasma high concentrated fibrinogen and thrombin does hold back human tumor cells and prevent pervasion.

It will further be appreciated by those skilled in the art and it is contemplated that variations to the embodiments illustrated and described herein may be made without departing from the spirit and scope of the present invention.

Accordingly, it is intended that the foregoing description is illustrative only, and the true spirit and scope of the invention will be determined by the appended claims.

See FIGS. 3A1, 3A2, 3B1, 3B2, 3C1 and 3C2.

Increased heat temperature intensify the density of the Glue membrane Various fold magnification has shown that we can't see any pore of FS gel membrane when is magnified at 3810 and 1600. At 27500 the pore of gel membrane is 0.005 micrometers and the heating temperature is at 101 oc.k

Electronic microscopic scanning results:
The pictures are taken under electronic microscopic with escalated magnification (up to 27.500-fold magnification). The corresponding scales under various magnifications are also marked. Basically we can't see any pore of FS gel membrane when is magnified at 3,810 and 1,600. At magnification of 15,000, we can see the pores of gel membrane about 0.1-0.2 um. At 27,500 magnification, we can see the pores of gel membrane less than 0.1 um in its diameter.
Magnification 27,500, the scale is 0.005 um in FIG. 4A.
Magnification 15,000, the scale is 1 um in FIG. 4B.
Magnification 3,810, the scale is 2 um in FIG. 4C.
Magnification 1,600, the scale is 10 um in FIG. 4D.
FIGS. 5A-5E are typical electronic microscopic scanning pictures at 27.500-fold magnification of FS gel membrane treated by different temperature. FIG. 5A is at 0° C. FIG. 5B is at 30° C. FIG. 5C is at 60° C. FIG. 5D is at 90° C. FIG. 5E is at 101° C.

A1AT can be detected in the high concentrated fibrinogen of FibrinGluRAAS®.

1. The existence of A1AT in the high concentrated fibrinogen of FibrinGluRAAS® can improve the stability of FS Glue membrane.

2. Neutralization of A1AT will accelerate the degradation of FS Glue membrane.

3. The further enrichment of A1AT in the high concentrated fibrinogen can greatly improve the stability of FS Glue membrane.

### EXAMPLES

1. In FIG. 6, A1AT is enriched in high concentrated fibrinogen of FibrinFluRAAS®.

The A1AT protein was detected by western blot using a polyclonal antibody against human A1AT. “TB” and “FNG” stand for the thrombin and high concentrated fibrinogen in FibrinGluRAAS®. TB was used for a negative control. “A1AT standard” stands for a commercially available A1AT as a positive control.

As can be seen from FIG. 7, the degradation of an FS glue membrane accelerated when A1AT in FNG is neutralized by an A1AT antibody. An A1AT polyclonal antibody was added to the high concentrated fibrinogen of FibrinGluRAAS®. “−” represents FibrinGluRAAS®+WFI, which means A1AT will keep its activity. “+” represents FibrinGluRAAS®+anti-A1AT antibody, which means A1AT is neutralized. The degradation of FS glue membranes is detected using SDS-PAGE (sodium dodecyl sulfate). When the A1AT is neutralized, the degradation of FS glue membranes begins as early as 15 minutes after the glue membrane is formed. The difference still exists at 2 hours, 4 hours, and 8 hours after the glue membrane is formed.

FIG. 8 shows that enrichment of A1AT in the purification process of high concentrated fibrinogen makes FS glue membrane much more stable. The degradation of FS glue membrane is compared between fibrinogen (FNG) and A1AT enriched high concentrated fibrinogen (QFNG). The degradation of FS glue membrane with A1AT enriched FNG will not begin within the first 8 hours after the gel is formed, which means it is much more stable.

FIG. 9 shows that the FS glue membrane can stay in a stable status as long as 48-72 hours using A1AT-enriched high concentrated fibrinogen. FIG. 9 shows the conditions of the membranes 48 hours after they are formed. The glue membrane is still in a stable status when using A1AT-enriched high concentrated fibrinogen (QFNG). However, the glue membrane of FNG has already degraded.

In Vitro Preliminary Study:

IN Vitro Studies of the Thrombin High Concentrate Fibrinogen and AFOD (HDL Apoa1) proteins containing good healthy cells destroyed all the solid tumor cancer cells.
Gastric cancer cell AGS in In Vitro Studies we found Thrombin a protein containing GOOD HEALTHY CELLS, High concentrate Fibrinogen another protein containing GOOD HEALTHY CELLS in combination of AFOD (HDL ApoA1) another protein with GOOD HEALTHY CELLS DESTROYED All remaining cancer cells in the surface areas where solid tumors have been removed (in reference to another patent application): application No. 61/457,380 filed on Mar. 14, 2011.
The following NINE CANCER CELLS LINES:

### 1. GASTRIC CELL LINES (AGS)

### 2. CERVICAL CANCER CELL LINE HELA

### 3. BREAST CANCER CELL LINE SK-BR-3

### 4. OVARIAN CANCER CELL LINE SK-OV-3

### 5. LUNG ADENOCARCINOMA CELL LINE SPC-A-1

### 6. ESOPHAGEAL CANCER CELL LINE TE-1

### 7. LIVER CANCER CELL LINE BEL-7402

### 8. PANCREASE CANCER CELL LINE PANC-1

### 9. LEUKEMIA CANCER CELL LINE DAMI

have been tested with Thrombin/High concentrated Fibrinogen and AFOD (HDL ApoAl) in preliminary INVITRO Studies at our R/D Lab and has been found that CANCER cells have been KILLED at certain level of protein concentration

### See FIG. 10.

In the table above the yellow color is the control medium with 0% of protein to compare with TB (Thrombin), FNG (Fibrinogen) and AFOD (HDL Apoa1)

A. TB (Thrombin)

### 1. Gastric Cell Lines (AGS)

See FIG. 11. Also see FIGS. 12A, 12B and 12C for Control, TB 5 U/mL and TB 25 U/mL, respectively.

### 2. Cervical Cancer Cell Line Hela

See FIG. 13. Also see FIGS. 14A, 14B and 14C for Control, TB 5 U/mL and TB 25 U/mL, respectively.

### 3. Breast Cancer Cell Line Sk-Br-3

See FIG. 15. Also see FIGS. 16A, 16B and 16C for Control, TB 5 U/mL and TB 25 U/mL, respectively.

### 4. Ovarian Cancer Cell Line Sk-Ov-3

See FIG. 17. Also see FIGS. 18A, 18B and 18C for Control, TB 5 U/mL and TB 25 U/mL, respectively.

### 5. Lung Adenocarcinoma Cell Line Spc-A-1

See FIG. 19. Also see FIGS. 20A, 20B and 20C for Control, TB 5 U/mL and TB 25 U/mL, respectively.

### 6. Esophageal Cancer Cell Line Te-1

See FIG. 21. Also see FIGS. 22A, 22B and 22C for Control, TB 5 U/mL and TB 25 U/mL, respectively.

### 7. Liver Cancer Cell Line Bel-7402

See FIG. 23. Also see FIGS. 24A, 24B and 24C for Control, TB 5 U/mL and TB 25 U/mL, respectively.

### 8. Pancrease Cancer Cell Line Panc-1

See FIG. 25. Also see FIGS. 26A, 26B and 26C for Control, TB 5 U/mL and TB 25 U/mL, respectively.

### 9. Leukemia Cancer Cell Line Dami

See FIG. 27. Also see FIGS. 28A, 28B and 28C for Control, TB 5 U/mL and TB 25 U/mL, respectively.

B. AFOD (HDL Apoa1)

### 1. Gastric Cell Lines (AGS)

See FIGS. 29A, 29B, 29C and 29D for Control, AFOD 0.1%, AFOD 0.5% and AFOD 2.5%, respectively.

### 2. Cervical Cancer Cell Line Hela

See FIGS. 30A, 30B, 30C and 30D for Control, AFOD 0.1%, AFOD 0.5% and AFOD 2.5%, respectively.

### 3. Breast Cancer Cell Line Sk-Br-3

See FIGS. 31A, 31B, 31C and 31D for Control, AFOD 0.1%, AFOD 0.5% and AFOD 2.5%, respectively.

### 4. Ovarian Cancer Cell Line Sk-Ov-3

See FIGS. 32A, 32B, 32C and 32D for Control, AFOD 0.1%, AFOD 0.5% and AFOD 2.5%, respectively.

### 5. Lung Adenocarcinoma Cell Line Spc-A-1

See FIGS. 33A, 33B, 33C and 33D for Control, AFOD 0.1%, AFOD 0.5% and AFOD 2.5%, respectively.

### 6. Esophageal Cancer Cell Line Te-1

See FIGS. 34A, 34B, 34C and 34D for Control, AFOD 0.1%, AFOD 0.5% and AFOD 2.5%, respectively.

### 7. Liver Cancer Cell Line Bel-7402

See FIGS. 35A, 35B, 35C and 35D for Control, AFOD 0.1%, AFOD 0.5% and AFOD 2.5%, respectively.

### 8. Pancrease Cancer Cell Line Panc-1

See FIGS. 36A, 36B, 36C and 36D for Control, AFOD 0.1%, AFOD 0.5% and AFOD 2.5%, respectively.

### 10. Leukemia Cancer Cell Line Dami

See FIGS. 37A, 37B, 37C and 37D for Control, AFOD 0.1%, AFOD 0.5% and AFOD 2.5%, respectively.

### Further In Vitro Studies:

In this study we want to demonstrate that in addition to the above nine cell lines we have added more different cells line at 0%, 2% and highest concentration of 10% IN ORDER TO COMPLETELY KILL ALL CANCERS CELLS.

In Vivo Study:

Fibrin Sealant (FibrinGluRAAS®) Plus a Slow-Released RAAS 1 to 45-FU Agent as a Prophylaxis for Peritoneal Dissemination of Gastric Cancer in Nude Mice Model

### OBJECTIVES

To evaluate the efficacy of Fibrin Sealant (FibrinGluRAAS®), used ALONE or combined with a slow-released RAAS 1 to 45 FU agent as an adjuvant instrument to prevent cancer cells from becoming detached and spreading into the abdominal cavity during the operation of gastrointestinal cancer.

### BACKGROUND

Gastrointestinal cancer still remains the most frequent malignancy worldwide. As far as the adeno-carcinoma of the stomach is concerned, it was the leading cause of cancer-related death worldwide through most of the twentieth century, and continues to be responsible for the majority of cancer deaths in developing countries. An estimated 875,000 new cases are diagnosed annually worldwide. Although the incidence of gastric cancer has decreased over the past century in some parts of the world, principally because of changes in diet, food preparation, and other environmental factors, yet, the declining trend has been limited to cancers below the esophagogastric junction. Furthermore, this incidence decline is being accompanied by a change in pattern toward more-aggressive variants of the disease. It is still estimated that 22,700 new cases are diagnosed annually in the United States, with approximately 11,800 deaths per year. In Asia and parts of South America, gastric cancer is the most common epithelial malignancy and leading cause of cancer-related death. In the Greater Shanghai Region of China, the number of newly diagnosed gastric cancer cases is over 6,000 each year.

Although combined therapies, including chemotherapy, radiation therapy and immunotherapy are performed in addition to surgical radical resection, nearly 50% of patients still die of recurrence, and a major form of recurrence was peritoneal dissemination. Concomitantly, peritoneal dissemination has been regarded as an important prognostic factor in GI cancer. It has also been indicated that increased peritoneal dissemination could result from the liberation, seeding and dissemination of cells from the primary tumor due to the surgical manipulation and mechanical tumor spillage. Moreover, the dissected and rough peritoneal surface may served as an appropriate “soil” for those iatrogenic tumor seeds. Besides strictly following the “non-touch” principle during the operation, it is absolutely necessary to develop new techniques and adjuvant equipment to abate unnecessary “nosocomial” tumor dissemination.

Shanghai RAAS Blood Products Company has recently observed that the glue membrane formed by lyophilized thrombin and lyophilized high concentrated fibrinogen (Fibrin Sealant FibrinGluRAAS®) can successfully prevent highly aggressive solid tumor cell lines, such as gastric cancer cell lines MKN45 and AGS, colon cancer cell line LoVo, as well as breast cancer cell line MCF-7, from passing through. The already finished in vitro study demonstrated the efficacy and potential role of FS as an adjuvant strategy in preventing solid tumor dissemination with the mechanism of creating a compact mechanical barrier.

Under electro-microscopy, the pore of glue membrane formed by Fibrin Sealant (FibrinGluRAAS®) was measured to be 0.6 μm or less in diameter, as little as 0.0 μm, while the size of solid tumor cell ranges from 10 to 100 μm. Theoretically, the dense and well-intertextured membrane formed by Fibrin Sealant can provide an optimal mechanical barrier to prevent the invasion and penetrating of tumor cells. As shown in FIG. 1, by using cell invasion assay (ECM550 kit, Chemicon International), it was found that the glue membrane formed by the Fibrin Sealant did prevent the above-mentioned high-aggressive tumor cell lines pervasion.

Besides such an exciting in vitro finding, the results of an in vivo study has also demonstrated that FS application can successfully prevent tumor cell dissemination intraperitoneally. A nude mice model was established to evaluate such efficacy.

Nude mice underwent laparotomy after anesthetization. The peritoneal surface was destroyed into multi-spots by using forceps, so as to create a congenial environment (“soil”) for the “seeding” of cancer cells. Tumor cells were sprayed into the abdominal cavity of mice in each group. Subsequently, Fibrin Sealant was applied to cover the peritoneal surface in the fibrin sealant group, while the control group were treated by normal saline only. After the glue membrane formed completely, the peritoneal cavity was closed. Two weeks after tumor cell implantation, peritoneal dissemination was identified macroscopically as small nodules growth on the surface of the small intestine and peritoneum, as well as the mesentery. A significantly larger number of peritoneal dissemination nodules were observed in the normal saline treatment group than in the FS treatment group. Furthermore, in the mice of self-control design, two weeks after tumor cells implantation, significantly larger and fused peritoneal cancer nodules were observed in the normal saline treatment side than in the Fibrin Sealant treatment side. And the above-mentioned in vivo data have been submitted for another international patent.

Herein, another idea has merged: whether or not the combination of FS with chemotherapeutic agent might reach a synergistic efficacy in preventing tumor cell dissemination.

Methodology (Study Design and Animal Models Development)

1. Preparation of Tumor Cells

Human gastric adenocarcinoma cells (cell line MKN45), were harvested from sub-confluent cultures (80-90% confluent) by a brief exposure to 1% trypsin. Trypsinization was stopped with a culture medium containing 10% fetal bovine serum, and the cells were washed twice by PBS and re-suspended in a serum-free medium. Only suspensions consisting of single cells with 90% viability were used for the experiments. The amounts of tumor cells for intra-peritoneal implantation were:

TABLE 1
Implanted tumor cells and amounts

| | Name of cell | | |
|---|---|---|---|
| Cancer type | line | Site of implantation | Implanted amounts |
| Stomach cancer | MKN45 | i.p. | 1 * 10⁷ per mouse |

These cells were in 0.2 ml serum-free DMEM medium for peritoneal surface coating.

2. Animals

Female Athymic nu/nu nude mice (NCI), at 6-7 weeks of age, weight ˜20 g, were purchased from the Shanghai Experimental Animal Center (License No. SYXK (Shanghai) 2003-0026), a Chinese animal facility with an international license. The mice were housed and maintained in a laminar airflow cabinet under specific pathogen-free conditions, and were housed for at least 7 days before use. All facilities used in this protocol were approved by the Animal Care and Use Committee of Shanghai Jiao-tong University.

3. Establishment of the Peritoneal Tumor Implanted Mouse Model

The mice were anesthetized with diethyl ether, and a simple laparotomy was performed via a central incision. The experiments were performed according to the following designs:

The mice were randomized classified into the following 4 groups: (1) FS group, (2) RAAS 1 to 45 FU group, (3) FS plus RAAS 1 to 45 FU group, and (4) control group. Mice in these 4 groups were underwent laparotomy after anesthetization. The peritoneal surface was destroyed into multi-spots by using forceps, so as to create a congenial environment (“soil”) for the “seeding” of cancer cells. Tumor cells were sprayed into the abdominal cavity of mice in each group. Subsequently, Fibrin Sealant, RAAS 1 to 45 FU, or both agents were applied to cover the peritoneal surface in FS group mice, RAAS 1 to 45 FU group mice, and combination group mice, respectively, while the control group were treated by normal saline only. After the glue membrane formed completely, the peritoneal cavity was closed.

4. Drug Formulation and Administration

Fibrin Sealant (FibrinGluRAAS®) is a two-component biological adhesive comprising a concentrated preparation of human fibrinogen and a concentrated preparation of human thrombin prepared from screened, pooled human plasma by Cohn fractionation with cold alcohol. Each kit contains 50-90 mg of high concentrated fibrinogen in 1 ml of water for injection and 500 IU of thrombin in 1 ml calcium chloride.

The individual components were prepared and withdrawn into syringes to which the provided mixing device was attached. Depressing the syringe plungers forced the component solutions into a mixing spray to form a thin layer.

5. End-Points Measurement

Two weeks after model establishment, the modified peritoneal cancer index (PCI), which is based upon the number and the size of the cancer nodules formed on the peritoneum and mesentery surface, was scored according to the following criteria.

As shown in FIG. 38, the peritoneal surface of the abdomen and pelvis were divided into 4 identified regions. For each region, a Lesion Size (LS) score was calculated for the largest tumor in that region (not the number of tumors in the area, just the size of the largest tumor in that particular region).
If there were no tumor nodules in a region, a score of zero was given to that region (LS-0).
If tumor nodules in a region were smaller than 2 mm, an LS score of one (LS-1) was given to that region.
If a region had tumor nodules from 4 mm present, it was given a lesion size score of two (LS-2).
If a region had tumor nodules greater than 5 mm or if it had tumors that converged (joined together), that section was given a score of three (LS-3).

FIG. 38 Shows Four Regions (Upper-Left, Upper-Right, Lower-Left, Lower-Right Quarters) of Peritoneal Surfaces of Abdomen

The lesion size scores for each of the 4 regions were added together. The highest score possible was 12 (4 times 3).

6. Mice Sacrifice and Sample Collection

All the mice were sacrificed using anesthesia overload. Peritoneal dissemination was assessed by the number of nodules larger than 1 mm in diameter and by the tumor volume score. Tumors were surgically removed and split in half at the indicated time points. One-half of the tumor was immediately snap-frozen in liquid nitrogen and stored at −80° C. until analysis. One-half of the tumor was fixed by 10% neutral buffered formalin (Richard-Allan Scientific, Kalamazoo, Mich.) for 24 hrs and embedded by paraffin.

Results

(1) Gross Appearance in the Peritoneal Surface:

Two weeks after tumor cell implantation, peritoneal dissemination was identified macroscopically as small nodules growth on the surface of the small intestine and peritoneum, as well as the mesentery. A significantly larger number of peritoneal dissemination nodules were observed in the normal saline treatment group than those in the other treatment groups (FIG. 39).

(2) Scores of Peritoneal Cancer Index (PCI) in FS Treatment Group and NS Group

The mean tumor volume score of PCI in control group was significantly higher than that in the other groups, with significant difference between groups (P<0.05).

### Conclusions

Herein, the results of both in vitro and in vivo studies demonstrated the efficacy and potential synergistic efficacy of FS combined with RAAS 1 to 45 FU, as an adjuvant strategy in preventing gastric cancer dissemination with the mechanism of creating a compact mechanical barrier and cytotoxic efficacy.

### FIG. 39 Intraperitoneal Tumor Growth in FS Treatment Group, Sino-Fuan Group, Combination Group and Control Groups in Gastric Cancer Models

A: in the control group, large and fused tumor growth was observed in NS treatment groups.
B: in FS treatment group, small, isolated and well-capsulized tumor nodules were found
C: in the Sino-fuan group, smaller nodules were found, and even some un-degraded agents were still left
D: in the combination group, tiny tumor nodules were found, synergistic anti-tumor efficacy of FS plus Sino-Fuan, a slow-released chemotherapeutic agent, was demonstrated in this group

### CONCLUSION

At the time of study when we have not discovered thrombin, high concentrate fibrinogen and AFOD (HDL ApoA1) containing GOOD HEALTHY CELLS at FIG. 39 above, Mice treated with ONLY FS small, isolated and well-capsulized tumor nodules were found and Gastric Tumor cells have been DESTROYED.

1. A method of purifying fibrinogen from plasma Fraction I or from plasma cryoprecipate to achieve better durable and better stability of fibrin sealant glue membrane, comprising
a) Collecting plasma cryopaste or Fraction I from human plasma;
b) Dissolving the Fraction I or cryopaste acquired in step a) in a pretreatment buffer, and conducting S/D virus inactivation for enveloped virus;
c) Loading the treated solution from step b) to a canion chromatography;
d) Using cold ethanol precipitation to purify fibrinogen from the flow through in step c);
e) Loading elution buffer I to obtain factor VIII;
f) Dissolving the paste in step d) by using Buffer II for final formulation; and
g) Dialyzing and adding stabilizer in the bulk acquired in step f).

2. The method of claim 1, wherein the Fraction I and cryopaste can be obtained by Cohn ethanol fractionation method.

3. The method of claim 1, wherein the pretreatment buffer includes Tris-HCl, NaCl-citrate, sucrose, and NaCl.

4. The method of claim 1, wherein a fibrinolysis inhibitor like A1AT is intentionally enriched and preserved during the process of fibrinogen of FibrinGluRAAS®.

5. The method of claim 1, wherein the existence of A1AT in the fibrinogen of FibrinGluRAAS® can improve the stability of FS Glue membrane.

6. The method of claim 1, wherein the further enrichment of A1AT in the fibrinogen can greatly improve the stability of FS Glue membrane.

7. A kit of lyophilized high concentrated fibrinogen and thrombin, wherein the high concentrated fibrinogen is intentionally enriched and preserved with the fibrinolysis inhibitor A1AT and either not heated or dry, wet or vapor heated up to at least 1° C., preferably at least 101° C. during the purification process of the high concentrated fibrinogen of FibrinGluRAAS® to intensify the stability and durability of the compound glue membrane, and the high concentrated fibrinogen undergoes 2 steps of virus inactivation for inactivating ALL enveloped viruses and a step of virus activation for inactivating ALL non-enveloped viruses.

8. The kit as claimed in claim 7, wherein the high concentrated fibrinogen and thrombin are sourced from human plasma tested Negative by NAT for HIV1-2, HBV, and HCV.

9. The kit as claimed in claim 7, wherein the A1AT is enriched and preserved during the purification process by dialysis to concentrate the A1AT.

10. The kit as claimed in claim 7, wherein the A1AT is enriched and preserved during the purification process by adding MORE pure A1AT to final bulk to maximize the stability and density of the compounded glue membrane.

11. The kit as claimed in claim 7, wherein the heating is done by the mean of Wet, Dry and Vapor up to at least 1° C. and above preferably at least 101° C. to intensify the density of the compounded glue membrane.

12. The kit as claimed in claim 7, wherein the step of virus inactivation for inactivating ALL non-enveloped viruses comprises heating up to at least 101° C.

13. The kit as claimed in claim 7, wherein the steps of virus inactivation for inactivating ALL enveloped viruses comprise solvent detergent (S/D) TNBP and Tween 80.

14. The kit as claimed in claim 7, wherein solvent detergent (S/D) TNBP and Tween 80 are applied for inactivating ALL enveloped viruses, and nanofiltration is applied for inactivating ALL non-enveloped viruses and ALL enveloped viruses in the thrombin.

15. The kit as claimed in claim 7, wherein the kit produces a compounded Glue membrane, the diameter of the mesh of which is smaller than human cancer cells, which are of the size of 10-100 μm. The actual size of which is 0.6 micrometer

16. The kit as claimed in claim 7, wherein the kit is applied in preventing dissociative tumor cell pervasion in clinical operations.

17. A method of preventing dissociative tumor cell pervasion in clinical operations comprising producing a GEL membrane on the SURFACES of AREAS where cancer tumors HAS BEEN REMOVED.

18. The method of claim 17, wherein the glue membrane is produced by applying a solution of lyophilized high concentrated fibrinogen and a solution of thrombin to the SURFACES of Areas where cancer tumor HAS BEEN REMOVED.

19. The method of claim 18, wherein the solution of high concentrated fibrinogen and the solution of thrombin are applied alternately to the SURFACES of Areas where cancer tumor HAS BEEN REMOVED.

20. The method of claim 18, wherein the solution of high concentrated fibrinogen and the solution of thrombin are each applied to the SURFACES of Areas where cancer tumor HAS BEEN REMOVED 3 to 5 times.

21. The method of claim 18, wherein both the high concentrated fibrinogen solution and the thrombin solution are applied to compound a glue membrane, the diameter of which is smaller than human cancer cells, which are of the size of 10-100 μm.

22. The method of claim 17, wherein the glue membrane can prevent cancer cells from becoming detached into the abdominal cavity during the surgical operation of gastrointestinal cancers in mice.

23. The method of claim 17 applied to all kinds of SOLID CANCER TUMORS which have not yet spread to the other parts of body, like solid cancers, AIDS related cancers, osteosarcoma, and cancers of the anus, appendix, bile duct, bladder, brain, breast, cervix, colon, esophagus, eye, gall bladder, head, neck, heart, liver, kidney, larynx, lip, oral cavity, lung, mouth, paranasal sinus and nasal cavity, ovaries, pancreas, parathyroid, penis, prostate, rectum, salivary glands, skin, spleen, throat, testicles, urethra, and vagina, as well as renal cell carcinoma.

24. The method of claim 17, wherein the compound glue membrane is APPLIED TOPICALLY, NOT INJECTED and NOT APPLIED for all blood (liquid) cancers, like leukemias, including acute myeloid leukemia (M0-M7), lymphoma, marrow malignancy, acute lymphoid leukemia (small, middle, large), myeloid disfunction syndrome (MDS), anemia, lupus and sclerosis in the brain. (Another RAAS ATBC1-9 agents for these applications under a different Patent application)

25. The method of claim 16, wherein the method inhibits the release of cytokines, including TNF (Tumor Necrosis Factor), and stops the activation of histones and toxicity produced by ONE TIME radio-chemotherapy to prevent the deaths of cancer patients due to the release of cytokines, histones, and toxicity produced by ONLY ONE TIME radio-chemo therapy IMMEDIATELY follow by the surgical operations to REMOVE CANCER TUMOR if Fribin Sealent (Fribin GluRAAS® is USED ALONE.

26. The method of claim 17 combining the glue membrane with a slow-release agent such as flourouracil (C4H3FN202) or any agents of RAAS 1 to RAAS 45 FU, as an adjuvant instrument to prevent cancer cells from becoming detached and spreading into the abdominal cavity during surgical operation of gastrointestinal cancer, and the flourouracil in the resultant glue membrane will KILL THE TUMOR CANCER CELLS; flouroucil is just one agent that can kill tumor cancer cells; other agents that can kill tumor cancer cells can be used instead of, or in addition to, flourouracil; the agents that can kill tumor cancer cells that are contemplated for use with the present invention include all presently known such agents:

| | Drugs which | Drugs which |
|---|---|---|
| Drugs which usually do | sometimes | usually don't |
| cause hair loss | cause hair loss | cause hair loss |
| Adriamycin | Amsacrine | Methotrexate |
| Daunorubicin | Cytarabine | Carmustine (BCNU) |
| Etoposide | Bleomycin | Mitroxantrone |
| Irinotecan (Campto) | Busulphan | Mitomycin C |
| Cyclophosphamide | 5 Fluorouracil | Carboplatin |
| Epirubicin | Melphalan | Cisplatin |
| Docetaxel, (Taxotere) | Vincristine | Procarbazine |
| Paclitaxel, (Taxol) | Vinblastine | 6-Mercaptopurine |
| Ifosphamide | Lomustine (CCNU) | Sreptozotocin |
| Vindesine | Thiotepa | Fludarabine |
| Vinorelbine | Gemcitabine | Raltitrexate (Tomudex) |
| Topotecan | | Capecitabine |

as well as all such agents that will become available in the future.

27. The method of claim 4, wherein the A1AT can be used as a stabilizer for fibrinogen, high concentrate fibrinogen, or any other product that needs the stability.

28. The method of claim 17, further comprising combining the glue membrane with an agent capable of killing tumor cancer cells.

29. The method of claim 28, wherein the agent is flourouracil.

30. The kit (FibrinGluRAAS) is used ALONE, Throat cancer patients WILL NOT LOOSE THEIR TASTE because No Chemo Radio Therapy is USED.

31. The kit (FibrinGluRAAS) is used ALONE will prevent SOLID TUMOR Cancer Patients from LOOSING THEIR HAIR during the treatment as No Chemo Radio Therapy is USED.

32. The kit claims the creation of Glue Membrane with a diameter less than 10 micrometers by other methodologies, by the use of any agents or materials from ANY sources (Animals, RDNA, etc to compound a Glue Membrane

33. The kit claims Thrombin a protein containing GOOD HEALTHY CELLS can KILL all solid Tumor cancers cells or Blood (Liquid) cancers cells

34. The kit claims High Concentrate Fibrinogen a protein containing GOOD HEALTHY CELLS can KILL all solid Tumor cancer cells or Blood (liquid) cancers cells.

35. The kit claims AFOD (HDL (ApoA1)) a protein containing GOOD HEALTHY CELLS can KILL all solid Tumor cancer cells or Blood (Liquid) cancers cells.

36. The kit claims Thrombin, High concentrate Fibrinogen which are proteins containing GOOD HEALTHY CELLS can KILL all solid Tumor cancer cells or Blood (Liquid) cancers cells.

37. The kit claims Thrombin, High concentrate Fibrinogen and AFOD (HDL ApoA1) which are proteins containing GOOD HEALTHY CELLS. With this combination of proteins which can MORE EFFECTIVELY KILL all solid Tumor cancer cells or Blood (Liquid) cancer cells.
